(19) United States
(12) Patent Application Publication
(10) Pub. No.: US 2007/0173923 A1
(43) Pub. Date: Jul. 26, 2007

(54) Drug reservoir stent

(76) Inventors: Douglas R. Savage, Del Mar, CA (US); Ronald E. Betts, La Jolla, CA (US)

Correspondence Address: PERKINS COIE LLP, P.O. BOX 2168, MENLO PARK, CA, 94026, US

(21) Appl. No.: 11/336,367

(22) Filed: Jan. 20, 2006

(51) Int. Cl. A61F 2/90; B05D 5/00

(52) U.S. Cl. 623/1.15; 623/1.42; 427/2.21

(57) ABSTRACT

Drug reservoir stents and methods of making and using the same are described. Such drug reservoir stents are prepared by applying a sacrificial material to one or more surfaces of the strut filaments of a drug delivery stent and applying a durable coating material to the surface of the sacrificial material to create a durable shell. A drug reservoir is created between the surface(s) of the strut filament and the durable shell by creating at least one perforation in the durable shell and removing the sacrificial material. The resulting reservoir is then filled with one or more therapeutic drugs. The drug reservoir stent allows elution of drug in the absence of a polymer binder.

### BACKGROUND

A stent is an endoprosthetic implant, usually generally tubular in shape, typically having a latticed, connected-wire tubular construction which is expandable to be permanently inserted into an anatomical lumen to provide mechanical support to the lumen and to maintain or to re-establish a flow channel within said lumen. For example, an endovascular stent may be inserted into a blood vessel during angioplasty, and is designed to prevent early collapse of a vessel that has been weakened and/or damaged by angioplasty. Insertion of endovascular stents has been shown to prevent negative remodeling and spasm of the vessel while healing of the damaged vessel wall proceeds over a period of months.

During the healing process, inflammation caused by angioplasty and stent implant injury often causes smooth muscle cell proliferation and regrowth inside the stent, thus partially closing the flow channel, and thereby reducing or eliminating the beneficial effect of the angioplasty/stenting procedure. This process is called restenosis. Blood clots may also form inside of the newly implanted stent due to the thrombotic nature of the stent surfaces, even when biocompatible materials are used to form the stent.

While large blood clots may not form during the angioplasty procedure itself or immediately post-procedure due to the current practice of injecting powerful anti-platelet drugs into the blood circulation, some thrombosis is always present, at least on a microscopic level on stent surfaces, and it is thought to play a significant role in the early stages of restenosis by establishing a biocompatible matrix on the surfaces of the stent whereupon smooth muscle cells may subsequently attach and multiply.

Stent coatings are known which contain bioactive agents that are designed to reduce or eliminate thrombosis or restenosis. Such bioactive agents are often dispersed or dissolved in either a bio-durable or bioerodable polymer matrix which is applied as a coating over the entire filament surface. After implantation, the bioactive agent diffuses out of the polymer matrix and preferably into the surrounding tissue.

If the polymer is bioerodable, in addition to release of the drug through the process of diffusion, the bioactive agent may also be released as the polymer degrades or dissolves, making the agent more readily available to the surrounding tissue environment. Bioerodable stents and biodurable stents are known where the outer surfaces or even the entire bulk of polymer material is porous. For example, PCT Publication No. WO 99/07308, which is commonly owned with the present application, discloses such stents, and is expressly incorporated by reference herein. When bioerodable polymers are used as drug delivery coatings, porosity is variously claimed to aid tissue ingrowth, make the erosion of the polymer more predictable, or to regulate or enhance the rate of drug release, as, for example, disclosed in U.S. Pat. Nos. 6,099,562, 5,873,904, 5,342,348, 5,873,904, 5,707,385, 5,824,048, 5,527,337, 5,306,286, and 6,013,853.

Heparin and other anti-platelet of anti-thrombolytic surface coatings are known which are chemically bound to the surface of the stent to reduce thrombosis. Stents have been described which are impregnated with both heparin and rapamycin, see U.S. Pat. No. 5,288,711 for example. U.S. Pat. No. 6,231,600 discloses that a mixture of polymer and therapeutic substance can be coated onto the surface of a stent, which is then coated with a second layer of polymer. The first layer may contain polymer mixed with a therapeutic substance and the second layer may contain polymer mixed with heparin.

A variety of agents specifically claimed to inhibit smooth muscle-cell proliferation, and thus inhibit restenosis, have been proposed for release from endovascular stents. As examples, U.S. Pat. No. 6,159,488 describes the use of a quinazolinone derivative; U.S. Pat. No. 6,171,609 and U.S. Pat. No. 5,716,981 describe the use of paclitaxel (taxol). Use of the metal silver is cited in U.S. Pat. No. 5,873,904. Tranilast, a membrane stabilizing agent thought to have anti-inflammatory properties is disclosed in U.S. Pat. No. 5,733,327. Rapamycin (sirolimus), an immunosuppressant reported to suppress both smooth muscle cell and endothelial cell growth, has been shown to have improved effectiveness against restenosis, when delivered from a polymer coating on a stent. See, for example, U.S. Pat. Nos. 5,288,711 and 6,153,252. Also, in PCT Publication No. WO 97/35575, the macrocyclic triene immunosuppressive compound everolimus and related compounds have been proposed for treating restenosis; see also WO 2003/090684, which is commonly owned with this patent application and are incorporated herein by reference. In U.S. Pat. No. 6,939,376, Shulze et al. disclose a stent for inhibiting restenosis which is comprised of a stent body and a bioerodable drug-release coating which contains poly(D,L-lactide) polymer and an immunosuppressive compound which is eluted with time at the vascular site of injury. U.S. Pat. No. 6,808,536 discloses local delivery of rapamycin or its analogs from an intravascular stent, either directly from tiny micropores or channels in the stent body or mixed or bound to a polymer coating applied on stent, grooves or channels which are smaller in dimension than the stent struts. Also, U.S. Pat. No. 6,904,658, “Process for Forming a Porous Drug Delivery Layer,” contains reference to the use of a porous plated layer to contain and elute therapeutic drug.

Given the proven advantages of implanting a stent designed to release a drug into lumenal tissue, it would be desirable to produce a drug-eluting stent having one or more additional advantages of (i) allowing a greater amount of drug to be “loaded” into the stent than when a surface drug coating is used, (ii) allowing elimination of polymer binders and other non-drug components that may cause irritation or inflammation at the stent site, (iii) providing greater control of drug-release rate once the stent is placed at the site, by controlling reservoir volume and perforation, (iv) being suitable for use with drugs and/or formulations which will not readily adhere to stent surface, (v) protecting the drug layer from damage when the stent is crimped to the balloon, (vi) protecting the drug layer from abrasion during drug delivery to the site of lesion, (vii) reducing friction by means of the favorable nature of the durable layer, thus enhancing ease of delivery compared to surface-coated stents, and (viii) facilitating application of drug to the stent, i.e., by dropping into holes as opposed to spray-coating. These and other advantages are provided by the drug reservoir stent of the instant invention.

### DESCRIPTION OF DRAWINGS

FIGS. 1A-1B are line drawings illustrating an endovascular stent having a metal-filament body and shown in contracted (1A) and expanded (1B) conditions.

FIG. 2 is a model showing a cross-section of one of the many filaments or “struts” having a perforated durable shell coating with a plurality of drug reservoirs which make up the body of a stent.

FIG. 3 is a line drawing illustrating a robotic delivery device for applying drug to a stent.

FIG. 4 is a line drawing showing a cross section of a stent of the invention placed at an intravascular site.

FIGS. 5A-5B are micrographs of the drug eluting stent of the present invention. FIG. 5A is a light photomicrograph taken at 80×, and FIG. 5B is a scanning electron micrograph taken at 150×. The array of perforations in the durable shell can be seen.

FIGS. 6A-6B are bar graphs showing vascular response to implantation with drug reservoir stents with and without the drug Biolimus A9. FIG. 6A shows the intimal thickness in microns of porcine coronary arteries after implantation with drug reservoir stents with or without drug. FIG. 6B shows percent area stenosis in porcine coronary arteries after implantation with drug reservoir stents with or without drug.

FIGS. 7A-7B are micrographs of sections of porcine coronary arteries after implantation of drug reservoir stents without (FIG. 7A) and with (FIG. 7B) Biolimus A9 loading.

### SUMMARY

In one aspect, the present invention provides a drug delivery stent for implanting in a bodily passageway, lumen or duct. The stent body is radially expandable and is formed of one or more metallic or polymer strut filaments, at least one surface of which is covered or coated by a perforated coating. A drug reservoir containing one or more therapeutic drugs is present between the stent surface and the perforated coating. The therapeutic drug may be an anti-restenosis drug, an anti-proliferative drug, an immunosuppressive compound, an antibiotic, an anti-thrombogenic drug or a cytotoxic compound. In some embodiments the drug is rapamycin, everolimus, paclitaxel, ABT-578, Biolimus A9, TRM-986, heparin, tranilast, beta-estradiol, or cyclosporin.

In another aspect, methods for forming drug delivery stents which contain drug reservoirs are described. Such stents are formed by applying a sacrificial material to at least one surface of each strut filament of a drug delivery stent, applying a coating material to the surface of the sacrificial material, thereby creating a durable coating, creating at least one perforation in the coating, and removing the sacrificial material to create a drug reservoir between the strut filament surface and the coating. The drug reservoir may be filled with a therapeutic drug.

### DETAILED DESCRIPTION

### I. Stent Geometry

Stents are generally comprised of filamentous structures called “struts” or “filaments” which are arranged in a generally tubular array and are expandable to provide support to vascular tissues. In viewing the stent from the end and through the tubular shape, each strut has an outer or exterior surface which faces the tissue of the body lumen into which the device is deployed. The inner or interior surface of each strut is the surface which is in contact with circulating blood or body fluids. Each strut also has two side surfaces connecting the outer and inner surfaces of the strut in the longitudinal direction (lengthwise along the strut) and two end surfaces connecting the outer and inner surfaces of the strut in a crosswise direction. Although the strut filaments are typically rectangular in cross section, it will be appreciated that they may be to some degree rounded or circular in shape.

### II. Drug Reservoir Stent

Stents according to the present invention have a stent body formed of one or more metallic or polymeric filaments or “struts.” These struts bear a perforated durable shell on at least one surface, typically the outer surface, i.e., the side intended to face the inner surface of a body lumen, with a drug reservoir located between the surface of the filament and the shell. The formation of this drug reservoir may be accomplished using a “sacrificial” material as will be described further below.

A. Stent body

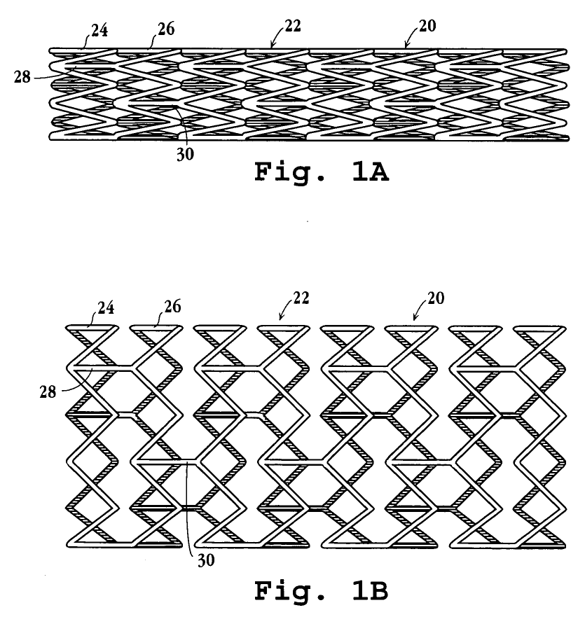

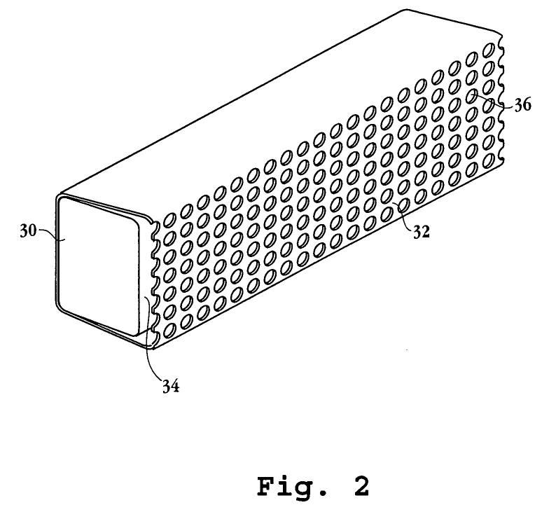

A stent is a type of endoprosthetic implant, usually generally tubular in shape, typically having a latticed, connected-wire tubular construction which is expandable to be permanently inserted into an anatomical lumen to provide mechanical support to the lumen and to maintain or to re-establish a flow channel within said lumen. For example, an endovascular stent may be inserted into a coronary artery during angioplasty to maintain patency of the blood vessel. Stents are also known for use in other blood vessels, such as the aorta or carotid artery, to treat arterial blockage or aneurysm, for example. In addition, stents are known for use in maintaining patency of body lumens or channels besides blood vessels; these include bile duct stents, urethral stents, and the like. The basic requirement of a stent body is that it be radially expandable upon deployment at the target site in the body, and that it has an open or latticed structure, allowing endothelial cell ingrowth through the gaps in the stent wall structure. The stent body is typically made up of linked filaments or “struts” which, in some embodiments, are made of cobalt-chromium alloys, stainless steel, platinum-iridium alloys or other biocompatible metals known in the art. In other embodiments, the stent body may be formed of biocompatible polymers, which may be bioerodable. Often these filaments form a zig-zag, sawtooth or sinusoidal wave structure, but other geometries such as a helical ribbon coil are also well known; for examples see U.S. Pat. No. 5,133,732 which discloses a continuous wire form having a deformable zig-zag pattern. In the embodiment shown in FIGS. 1A-1B, the stent body is formed of a plurality of linked tubular members by filaments, such as members 24, 26. Each member has an expandable zig-zag, sawtooth, or sinusoidal wave structure. The members are linked by axial links, such as links 28, 30 joining the peaks and troughs of adjacent members. As can be appreciated, this construction allows the stent to be expanded from a contracted condition, shown in FIG. 1A, to an expanded condition, shown in FIG. 1B, with little or no change in the length of the stent. Also included are helical ribbon designs, for example those disclosed in U.S. Pat. No. 6,899,730, which is incorporated by reference herein. Many other examples of stent designs are known in the art.

B. Stent Drug Reservoir and Coating

FIG. 2 is a cross-section of one of the many filaments or “struts” which make up the stent body. As seen in FIG. 2, the stent filament 30 is coated with a coating 32, which is preferably a durable shell or coating, having at least one perforation 36 over a drug reservoir 34. In this embodiment, the filament 30 has a roughly rectangular in cross-section, although it will be appreciated that other geometries are contemplated. Also shown is the durable shell 32 having at least one perforation 36 communicating with a drug reservoir 34 located between the outside surface of the strut and the shell. The durable shell is continuous over at least one strut surface, typically the outer surface, and contains one or more perforations or holes 36 which may be arranged in a desired pattern or density. In a preferred embodiment, the perforations extend the entire width of the durable shell thus to form a plurality of holes in the shell coating that contact the reservoir 34. These perforations allow construction of the drug reservoir as is described below, allow introduction of drug into the reservoir, and allow diffusion of the drug from the reservoir (i.e., the stent) to the surrounding tissue upon deployment.

In one embodiment, the drug reservoir of the stent is fabricated by removal of a sacrificial material layer to create an open space or reservoir limited by the exterior surface of the stent filament and the shell, which is preferably formed of a durable material. The sacrificial material, which in one embodiment is a formulation of a polymer such as poly (D,L-lactide) in a suitable solvent such as acetone, is spray-coated on the outer surface of a filament which is part of a stent. Depending on the geometry of the filaments of the stent, the desired tissue contact and/or amount of drug loaded, one or both of the side surfaces and/or the inner surface of each filament may be coated in addition to or instead of the outer surface. The sacrificial coating may be applied by spray means or manual application, e.g., using a capillary. Typically the stent body is coated in order to coat the exposed outer (and optionally side or inner) surface(s) of substantially every filament. By “substantially” it is preferred that at least 85-100% of each filament has at least one surface coated. In a preferred embodiment, at least 95-100% of the filaments have at least one surface coated. Preferably at least 95-100% of at least one of the exposed outer, side or inner surface of each filament is coated with the sacrificial material. While poly(D,L-lactide) is a preferred sacrificial material, it will be appreciated that other materials may be used which fit the desired characteristics, particularly that they may be readily applied to the outer surface of the filament and then easily or readily removed once the durable shell has been applied. Exemplary sacrificial materials include glucose, lactose, dextrose or sodium chloride applied in water solution; polymethylmethacrylate or polyvinylchloride applied in methylene chloride; polyurethane in xylene or toluene, and glycolic acid/lactic acid copolymers (PGLA polymers) in acetone. In embodiments of the invention, the sacrificial material may be laid down in multiple coats or as a single layer. It should be appreciated that the thickness of the sacrificial material is determined by the total volume of sacrificial material applied, and this thickness will determine the volume of the drug reservoir which results from removal of the sacrificial polymer in later steps. Varying the reservoir volume allows control over drug dosing. For example, a reservoir thickness (depth) of about 3-50 microns is suitable for a drug eluting stent application. In a preferred embodiment, the reservoir thickness is about 3-30, preferably about 10 microns. It will be appreciated that the reservoir thickness may be adjusted to vary the volume of the reservoir and the amount of drug available. The reservoir volume may be calculated using the reservoir thickness and the area of the stent surface. For example, with a reservoir thickness of 10 microns and an area of the entire outer surface of the stent of about 25 mm², the reservoir volume capable of storing drug would be approximately 0.010 mm×25 mm² or 0.25 mm³ (25 μl liquid volume). Reducing the amount of sacrificial material to a thickness of 8 microns reduces the reservoir volume by approximately 20%. Conversely, increasing the reservoir thickness (or the area of the stent surface) increases the reservoir volume.

In one embodiment of the invention, a formulation of 25 mg of poly(D,L-lactide) in acetone is spray coated on the outer and side surfaces of the metallic filaments of an endovascular stent. The spray coating may be accomplished using ultrasonication techniques enabling the dissolved polymer to be atomized into fine fragments or droplets of approximately between 4 and 100 microns. A suitable nozzle available from Sonotek Inc. (Milton, N.Y.) is driven by an Rf generator at 50 MHz and a compressed air column of flow rate 1 to 5 cubic feet per minute may be used to atomize and disperse the mixture onto the stent surface in a single application. The coated stent is then dried such as with a vacuum chamber at −27 inches mercury gauge pressure for a period sufficient to eliminate the solvent component, typically about four hours or more.

A conformal coating of a durable material is applied to the coated stent surface, i.e., to the surface of the sacrificial material coating on the stent, to form a durable shell. In one embodiment, the conformal coating is poly-para-xylylene, commonly known in the art by the trade name, “parylene,” which may be applied to the coated stent surface by condensation of the para-xylylene dimer. Although the parylene layer may be applied in a wide range of thicknesses, a coating thickness that allows the completed shell structure to be self-supporting is preferred. In some embodiments, the thickness of the parylene layer is about two to about twenty microns, preferably about 10 microns. In an embodiment, a parylene C thickness of about ten microns is used. The durable material may be laid down in multiple coats or as a single layer. In a preferred embodiment, the durable material is applied in multiple coats. Parylene C coatings may be obtained from Advance Polymers, Rancho Cucamonga, Calif., and SCS Polymers of Indianapolis, Ind. Those familiar with this material will appreciate that there are other suitable parylenes for use in accordance with the present invention, including trade names parylene C, parylene N, and parylene D, which may be used alone or in combination to form the durable shell.

The durable shell preferably includes at least one perforation or hole in order for the sacrificial material to be removed, forming the drug reservoir. It will be appreciated that the perforation may be formed on any surface of the durable shell. In a preferred embodiment, a plurality of perforations are formed on the outer (tissue contacting surface) of the durable shell. In one embodiment, the durable shell (e.g., parylene) is perforated by means of an excimer laser (Spectralytics, Dassel, Minn.), forming at least one perforation which penetrates the durable shell completely, exposing the sacrificial polymer. The diameter of the perforation is preferably from about 2 to about 20 microns, but any hole diameter is comprehended by the present invention provided that the resulting porosity of the shell is sufficient for removal of the sacrificial material (see details below), to allow the introduction of drug into the drug reservoir, and/or to allow diffusion of drug into bodily tissue. Preferably there are a plurality of perforations, substantially all of which penetrate the durable shell completely. In one embodiment, these holes may be arranged apart from each other by orthogonal distances of from about 5 to about 50 microns and may form a geometric array. The perforations may be formed in the durable shell in any suitable density or pattern. As will be appreciated, the size and number of perforations may be varied to control drug dosage and rate of diffusion.

Following perforation of the durable shell, the sacrificial material is preferably removed, creating the void space which constitutes the drug reservoir. In the embodiment so far described, the poly(D,L-lactide) sacrificial material is removed from the stent by immersing the stent in solvent to dissolve the sacrificial layer, leaving an empty cavity which is used as the drug reservoir. In one embodiment, the stent is soaked in 100% acetone for 15 minutes to dissolve the sacrificial layer such as poly(D,L-lactide) polymer. The resulting drug reservoir stent is then loaded with a therapeutic drug.

C. Drug Reservoir Stent Loading

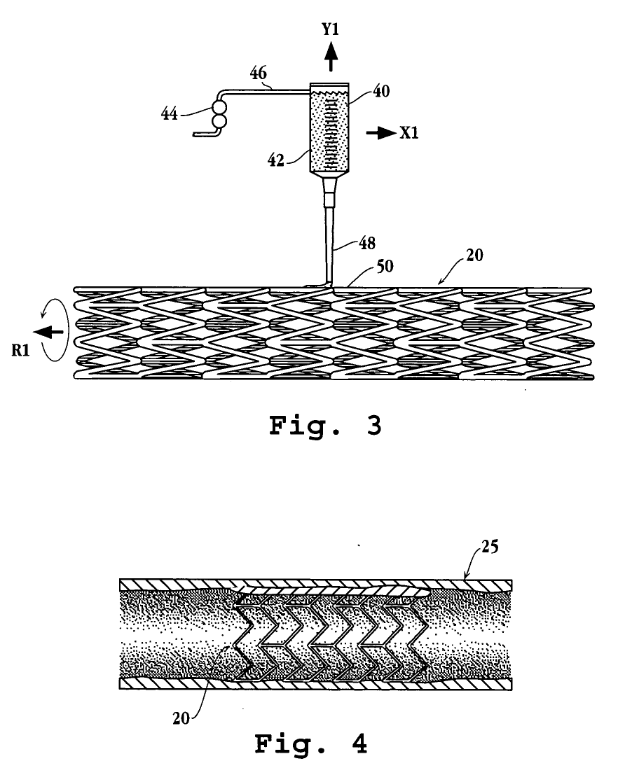

According to the present invention, drug reservoir stents are designed to release one or more therapeutic drugs. Examples of types of drugs useful for application via stent include anti-restenosis drugs, anti-proliferative drugs, immunosuppressive compounds, antibiotics, anti-thrombogenic drugs and cytotoxic compounds. In preferred embodiments the drug is an anti-restenosis anti-proliferative drug such as rapamycin (sirolimus), everolimus, paclitaxel, ABT-578 (a rapamycin-like agent that binds the FKBP12 protein), Biolimus A9 (an everolimus derivative), TRM-986, heparin, tranilast, beta-estradiol, or cyclosporin. Therapeutic drugs may be introduced into the stent reservoir by direct loading, e.g., by direct application through the perforations in the durable coating into the drug reservoir, e.g., with a capillary or hypodermic needle and syringe, by inkjet injection or by immersion of all or part of the stent in a drug solution. The drug reservoir stent is then said to be “loaded” or “saturated” with drug. In some embodiments, a substantially inert binder may also be introduced into the reservoir in addition to the therapeutic drug(s). Examples of substantially inert binders are poly(DL-lactide) polymer and poly(L-lactide). In one embodiment, the drug is incorporated in the inert binder prior to being loaded in the reservoir. By way of example, FIG. 3 illustrates a robotic device useful in depositing the drug into the reservoir of a stent filament (reservoir not shown). A drug solution or mixture 40 is made by dissolving the drug in a suitable solvent. The viscosity of the solvent mixture may be adjusted by varying the amount of solvent, and it ranges from 2 centipoise to 2000 centipoise. If desired, polymer molecules may be added to increase solution viscosity.

The drug solution is placed in a pressurizable reservoir 42. Connected to the reservoir is a fluid pressurization pump 44. The pressurization pump may be any source of pressure capable of urging the solvent mixture to move at a programmed rate through a solution delivery tube 46. The pressure pump 44 is under the control of a microcontroller (not shown), as is well known in the field of precision dispensing systems. For example, such a microcontroller may comprise 4-Axis Dispensing Robot Model numbers I&J500-R and I&J750-R available from I&J Fisnar Inc, of Fair Lawn, N.J., which are controllable through an RS-232C communications interface by a personal computer, or precision dispensing systems such as Automove A-400, from Asymtek, of Carlsbad, Calif. A suitable software program for controlling an RS232C interface may comprise the Fluidmove system, also available from Asymtek Inc.

Attached to reservoir 42, for example, at the bottom of the reservoir, is a solution delivery tube 48 for delivery of the solvent mixture to the surface or drug reservoir of the stent. The pressurizable reservoir 42 and delivery tube 48 are mounted to a moveable support (not shown) which is capable of moving the solvent delivery tube in small steps such as 0.2 mm per step, or continuously, along the longitudinal axis of the stent as is illustrated by arrow X1. The moveable support for pressurizable reservoir 42 and delivery tube 46 is also capable of moving the tip (distal end) of the delivery tube closer to the microfilament surface or up away from the microfilament surface in small steps as shown by arrow Y1.

The stent is gripped by a rotating chuck contacting the inner surface of the stent at least one end. Axial rotation of the stent can be accomplished in small degree steps, such as 0.5 degree per step, to reposition the uppermost surface of the stent structure for access by the delivery tube by attachment of a stepper motor to the chuck as is well known in the art. The chuck and stepper motor system may be purchased from Edmund Scientific of Barrington, N.J. If desirable, the stent can be rotated continuously. The method of precisely positioning a low volume fluid delivery device is well known in the field of X-Y-Z solvent dispensing systems and can be incorporated into the present invention. Alternatively, the delivery tube can be held at a fixed position and, in addition to the rotation movement, the stent is moved along its longitudinal direction.

The action of the fluid pressurizing pump, X1 and Y1 positioning of the fluid delivery tube, and R1 positioning of the stent are typically coordinated by a digital controller and computer software program, such that the precisely required amount of solution is deposited wherever desired on or in the reservoir of the stent.

The X-Y-Z positioning table and moveable support may be purchased from I&J. Fisnar. The solution delivery tube preferred dimensions are preferably between 18-28 gauge stainless steel hypotubes mounted to a suitable locking connector. Such delivery tubes may be obtained from EFD Inc of East Providence, R1. See EFD's selection guide for Special Purpose Tips. The preferred tips are reorder #'s 5118-1/4-B through 5121-1/4-B “Burr-free passivated stainless steel tips with ¼″ length for fast point-to-point dispensing of particle-filled or thick materials”, reorder #'s 51150VAL-B “Oval stainless steel tips apply thick pastes, sealants, and epoxies in flat ribbon deposits”, and reorder #'s 5121-TLC-B through 5125-TLC-B “Resists clogging of cyanoacrylates and provides additional deposit control for low viscosity fluids. Crimped and Teflon lined”. A disposable pressurizable solution reservoir is also available from EFD, stock number 1000Y5148 through 1000Y 5152F. An alternate tip for use with the invention is a glass micro-capillary with an I.D. of about 0.0005 to 0.002 inch, such as about 0.001 inch, which is available from VWR Catalog No. 15401-560 “Microhematocrit Tubes”, 60 mm length, I.D. 0.5-0.6 mm. The tubes are further drawn under a Bunsen burner to achieve the desired I.D. for precise application of the drug/solvent mixture. The programmable microcontroller to operate the stepper motor, and XYZ table is available from Asymtek, Inc. It is within the scope of the invention to use more than one of the fluid dispensing tube types working in concert, or alternately to use more than one moveable solution reservoir equipped with different tips, or containing different viscosity solutions or different chemical makeup of the multiple solutions in the same process.

### III. Methods of Use and Performance Characteristics

This section describes vascular treatment methods in accordance with one embodiment of the invention, and the performance characteristics of stents constructed in accordance with the invention.

The methods of using the drug reservoir stent as described below are intended to provide local drug administration to the interior of a bodily lumen. In one embodiment, the methods of the invention are designed to minimize the risk and/or extent of restenosis in a patient who has received localized vascular injury, or who is at risk of vascular occlusion. Typically the vascular injury is produced during an angiographic procedure to open a partially occluded vessel, such as a coronary or peripheral vascular artery. In the angiographic procedure, a balloon catheter is placed at the occlusion site, and a distal-end balloon is inflated and deflated one or more times to force the occluded vessel open. This vessel expansion, particularly involving surface trauma at the vessel wall where plaque may be dislodged, often produces enough localized injury that the vessel responds over time by inflammation, cell proliferation leading to positive remodeling, and reocclusion. Not surprisingly, the occurrence or severity of this process, known as restenosis, is often related to the extent of vessel stretching and injury produced by the angiographic procedure. Particularly where overstretching is 35% or more, restenosis occurs with high frequency and often with substantial severity, i.e., vascular occlusion.

In practicing the present invention, the stent is placed in its contracted state typically at the distal end of a catheter, either within the catheter lumen, or in a contracted state on a distal end balloon. The distal catheter end is then guided to the injury site, or the site of potential occlusion, and released from the catheter, e.g., by using a trip wire to release the stent into the site if the stent is self-expanding, or by expanding the stent on a balloon by balloon inflation, until the stent contacts the vessel walls, in effect, implanting the stent into the tissue wall at the site. Once deployed at the site, the stent immediately begins to release active compound into the cells lining the vascular site, to inhibit cellular proliferation. FIG. 4 shows the placement of a stent 20 at an intravascular site of injury in a vessel 25. The figure shows the stent in its expanded condition, after delivery to the site in a contracted condition, and radial expansion to an extent that presses the drug delivery reservoir stent body filaments against the walls of the vessel. This placement anchors the stent within the vessel and brings the outer surface of the stent into direct contact with the tissues lining the vessel, for drug delivery directly from the drug reservoir to the cells lining the vessel.

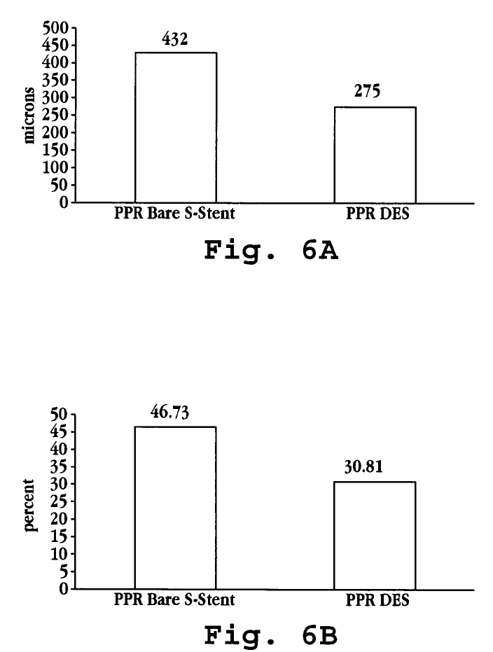

As described in Example 1, experiments were conducted in support of the invention with stents having an empty reservoir as compared to stents having a reservoir with Biolimus A9. FIG. 6A shows intimal thickness of porcine coronary arteries after implantation with drug reservoir stents including Biolimus A9 as a drug (here designated “PPR DES” for porous parylene reservoir drug eluting stent) or drug reservoir stents without drug (designated “PPR bare stent”). As seen in the figure, the stents including the vessels implanted with the PPR DES stents showed a decrease in the intimal thickness as compared to the vessels implanted with PPR DES bare stents. FIG. 6B shows the percent area stenosis in porcine coronary arteries after implantation with PPR DES stents or PPR bare stents. The intimal thickness for the PPR DES stents was 275 microns as compared to 432 microns for the PPR bare stents. Thus, the intimal thickness was over 35% less with the PPR DES stents as compared to the PPR bare stents. As seen in FIG. 6B, the percent area of stenosis was also remarkably decreased for the PPR DES stents (30.81%) as compared to the PPR bare stents (46.73%).

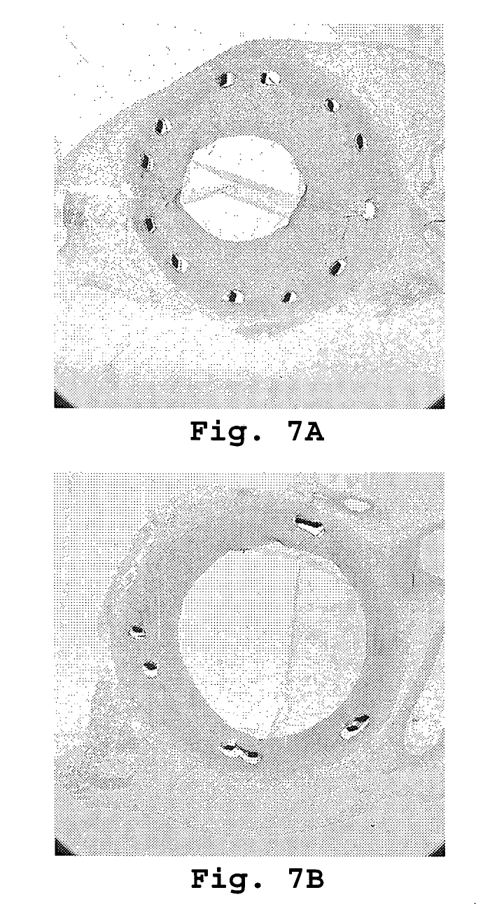

Further, the occlusion of vessels of pigs implanted with stents including the drug (FIG. 7B) was remarkably decreased as compared to stents having a reservoir, but no drug (FIG. 7A).

Polymeric stent coatings are known to produce increased vessel wall inflammation and restenosis, and the absence of polymeric components reduces inflammation and irritation at the vessel site, which can be caused, for example, by inflammatory cell reaction to breakdown of a biodegradable polymer or foreign body response to a stable polymer. At the same time, the drug reservoir and perforated durable shell of the current invention allow for greater amounts of drug loading into the stent, and greater control of drug dosage and release, than with surface-coated stents. This durable coating also protects the underlying drug from damage or abrasion (during delivery to the lesion or during crimping), and reduces friction during delivery, due to the favorable characteristics of the preferred coating, i.e., parylene.

Although the invention has been described with respect to particular embodiments and applications, it will be appreciated that various changes and modifications may be made without departing from the invention.

### IV. EXAMPLES

The following examples illustrate various aspects of making and using the stent invention herein. They are not intended to limit the scope of the invention.

### Example 1

A. Preparation of Drug Biolimus A9 in Acetone Solution

The therapeutic compound Biolimus A9 (42-O-(2-ethoxyethyl) rapamycin, CAS 851536-75-9, was evaluated in an animal model and delivered to vascular tissue using a drug reservoir stent of the instant invention. Biolimus A9 is an immunosuppressive, anti-proliferative compound. In this experiment the drug was completely dissolved in the solvent acetone in preparation for deposition onto the modified stent

B. Preparation of Drug Reservoir Stent Containing Biolimus A9

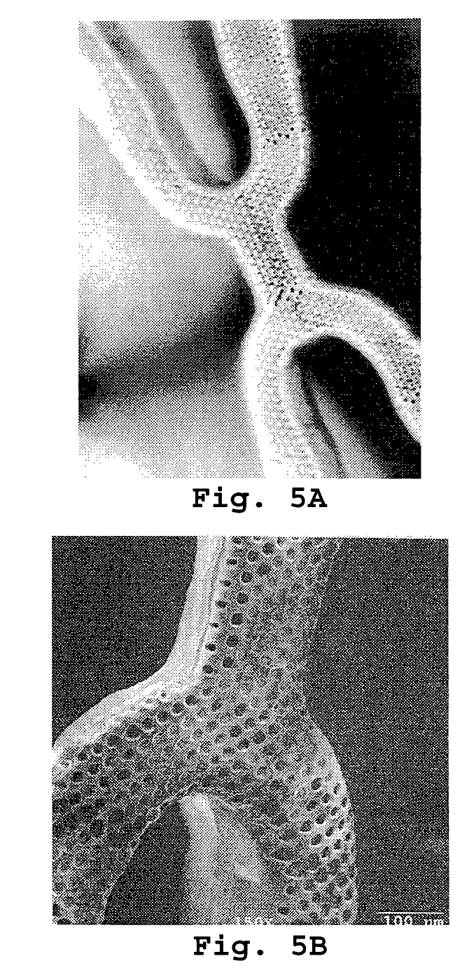

S-Stents, 15 mm length, from Biosensors Inc. (Newport Beach Calif.) were coated with poly(D,L-lactides) polymer from Sigma-Aldrich Inc. (St. Louis, Mo.) dissolved completely in acetone in a concentration of 25 mg/ml as a sacrificial layer. The stents were placed on an appropriate coating mandrel and coated using a 60 kHz Accu-mist™ brand ultrasonic nozzle made by Sono-Tek Inc. (Milton, N.Y.). The stents were then dried in a vacuum chamber at −29 inches Hg for 4 hours. The stents were coated with a 5 microns thick layer of parylene C polymer. These stents were then perforated with an excimer laser. The laser drilled holes were approximately 13 microns in diameter, spaced a distance of approximately 18 microns apart (on center). The perforations were primarily formed on the outer surfaces of the coated struts, as can be seen in FIGS. 5A-5B. The stents were then fully immersed in 100% acetone solvent for a period of one hour to dissolve the underlying sacrificial polymer. The prepared and dissolved Biolimus A9 was then applied to the stent surface by deposition through a blunt hypodermic needle attached to a 10 μl glass Hamilton HPLC syringe such that the reservoir space between the parylene C shell and the outer stent surface was saturated with the drug. The stents were dried in a vacuum chamber at −29 inches Hg for four hours to remove the solvent from the drug/solvent mixture. After drying, the stents were weighed to determine the drug content (dosage) per stent, averaging about 1000 micrograms per stent. The stents were then mounted with a crimping device to Senso balloon catheters from Biosensors International, using standard methods. The stent and delivery devices were then sterilized with electron beam sterilization at 25KGray.

C. Animal Implant Tests-Pilot Study

Six of the drug-saturated reservoir stents and six reservoir stents with no drug (the control group) to evaluate the vascular response of pigs implanted with 3.0 mm×15 mm porous parylene reservoir stents loaded with Biolimus A9 drug and no polymer binder. The stents were implanted into the coronary arteries of six pigs per standard research practice, as generally described by Schwartz et al. (“Restenosis After Balloon Angioplasty-A Practical Proliferative Model in Porcine Coronary Arteries”, Circulation 82:(6) 2190-2200, December 1990.). Each pig was implanted with one drug-saturated reservoir stent and one reservoir stents with no drug. Vascular response was evaluated at 28 days post-implant by vascular histopathological/morphometric analysis techniques. The bare (non-drug) reservoir stent was used as a baseline control. The drug dosage within the stent reservoir was approximately 225 micrograms. At this dosage, the drug-eluting stent would be expected to reduce intimal hyperplasia at 28 days.

At the termination of the experiment the stented arteries were harvested and prepared for histomorphometric analysis. Upon analysis the vascular response was evaluated. Results were quantitated and are shown as graphs in FIGS. 6A-6B. The control reservoir stents, which did not contain drug, produced a vascular response to the implant displayed as a mean new intimal thickness growth of 432 microns. The drug stents displayed a mean of only 275 microns of new intimal growth. The percent area stenosis of the non-drug control group was 46.73% and the percent area stenosis of the drug group was 30.81% (percent area stenosis=neointimal area/stent area×100). A representative histological micrograph is shown in FIGS. 7A-7B. The significant reduction in stenosis with the drug-loaded stent compared to the control stents strongly suggests that a beneficial effect was produced by the therapeutic drug Biolimus A9 when administered via the drug reservoir stent of the present invention.

We claim:

1. A drug delivery stent for implanting in a bodily passageway, lumen or duct, comprising:
a radially expandable stent body formed of one or more metallic or polymer strut filaments, each strut filament having outer, inner, side and end surfaces;
a drug reservoir composed of a therapeutic drug being carried on one or more surfaces of each strut filament; and
a durable shell coating covering said drug reservoir and having at least one perforation extending through the coating.

2. The stent of claim 1 wherein said drug reservoir has a thickness of between 2 and 30 microns.

3. The stent of claim 1 wherein the therapeutic drug is selected from the group consisting of an anti-restenosis drug, an anti-proliferative drug, an immunosuppressive compound, an antibiotic, an anti-thrombogenic drug and a cytotoxic compound.

4. The stent of claim 3 wherein the therapeutic drug is rapamycin, everolimus, paclitaxel, ABT-578, Biolimus A9, TRM-986, heparin, tranilast, beta-estradiol, or cyclosporin.

5. The stent of claim 1 wherein said durable shell coating comprises a polymer.

6. The stent of claim 5 wherein said polymer is parylene.

7. The stent of claim 1 wherein said reservoir further comprises a substantially inert binder.

8. The stent of claim 1 wherein said substantially inert binder is a polymer comprising poly(DL-lactide) or poly(L-lactide).

9. The stent of claim 1 wherein said shell covers at least the outer surface of substantially every filament of the stent.

10. A method of forming a drug delivery stent comprising the steps of:
applying a sacrificial material to at least one surface of each strut filament of a stent body formed of one or more filaments;
applying a durable coating material to said surface of said sacrificial material, creating a durable shell;
creating at least one perforation in said durable shell, and removing said sacrificial material to form a continuous reservoir disposed between said strut filament surface and said durable shell.

11. The method of claim 10 wherein said sacrificial material is applied substantially to the outer surface of each strut filament.

12. The method of claim 10 wherein said sacrificial material is applied substantially to the outer and side surfaces of each strut filament.

13. The method of claim 10 wherein said durable coating material comprises a polymer.

14. The method of claim 13 wherein said polymer is parylene.

15. The method of claim 10 wherein said sacrificial material comprises poly(D,L-lactide).

16. The method of claim 10 further comprising the step of filling said reservoir with a drug substance.
